(19) United States
(12) Patent Application Publication
(10) Pub. No.: US 2015/0190262 A1
(43) Pub. Date: Jul. 9, 2015

(54) STRAPS FOR DEVICES AND METHODS THEREFOR

(71) Applicant: Boa Technology Inc., Denver, CO (US)

(72) Inventors: James Capra, Steamboat Springs, CO (US); Tamara White, Denver, CO (US); Robert Burns, Denver, CO (US); Mark Kerns, Golden, CO (US); Mark Dutton, Steamboat Springs, CO (US)

(21) Appl. No.: 14/593,999

(22) Filed: Jan. 9, 2015

Related U.S. Application Data
(60) Provisional application No. 61/925,584, filed on Jan. 9, 2014.

(51) Int. Cl. A61F 5/01

(52) U.S. Cl. CPC A61F 5/0123; A61F 2250/001

(57) ABSTRACT

According to an embodiment, a strap includes a closure system and a strap system. The closure system includes a tension member, a tightening mechanism that tensions the tension member and maintains the tension member's tension, and a guide that routes the tension member between the guide and the tightening mechanism. Tensioning of the tension member causes the guide to move toward the tightening mechanism. The strap system includes a strap guide(s), a first end that is coupled to the tightening mechanism, a second end that is coupled with the guide, and a strap body. The strap body is positioned through the strap guide(s) so that at least a portion of the strap overlaps and so that the strap body forms a loop. Tensioning of the tension member reduces a diameter of the strap system's loop, which effects constriction of the strap system about an article disposed within the loop.

### CROSS-REFERENCES TO RELATED APPLICATIONS

This application claims priority to U.S. Patent Application No. 61/925,584 filed Jan. 9, 2014, entitled “Straps for Devices and Methods Therefor,” the entire disclosure of which is hereby incorporated by reference, for all purposes, as if fully set forth herein.

### BACKGROUND OF THE INVENTION

The present invention is related to closure devices for various articles, such as braces, medical devices, shoes, clothing, apparel, and the like. Such articles typically include closure devices that allow the article to be placed and closed about a body part. The closure devices are typically used to maintain or secure the article to the body part. For example, shoes are typically placed over an individual's foot and lace is tensioned and tied to close the shoe about the foot and secure the shoe to the foot. Conventional closure devices have been modified in an effort to increase the fit and/or comfort of the article about the body part. For example, shoe lacing configurations and/or patterns have been modified in an attempt to increase the fit and/or comfort of wearing shoes. Conventional closure devices have also been modified in an effort to decrease the time in which an article may be closed and secured about the body part. These modifications have resulted in the use of various pull cords, straps, and tensioning devices that enable the article to be quickly closed and secured to the foot.

### BRIEF SUMMARY OF THE INVENTION

The embodiments described herein provide straps that may be used closure systems to close and/or tighten an article, such as a brace, that is fit about a limb of the user. According to one aspect, a strap for tightening an article about a limb includes a closure system and a strap system. The closure system includes a tension member, a tightening mechanism that is configured to tension the tension member and to maintain the tension of the tension member, and a guide that routes or guides the tension member along a path between the guide and the tightening mechanism. Tensioning of the tension member pulls the guide toward the tightening mechanism.

The strap system includes one or more strap guides, a first end that is coupled to the tightening mechanism, a second end that is coupled with the guide, and a strap body that is positioned between the first and second ends. The strap body is disposed through the one or more strap guides so that at least a portion of the strap body overlaps on itself and so that the strap body forms a loop between the first and second ends. Tensioning of the tension member via the tightening mechanism causes the loop of the strap system to reduce in diameter, which effects constriction of the strap system about an article that is disposed within the loop.

According to another aspect, a method for tightening an article about a limb includes providing a strap that is configured to tighten the article about the limb. The strap includes a closure system and a strap system. The closure system includes a tension member, a tightening mechanism that is configured to tension the tension member and to maintain the tension of the tension member, and a guide that routes or guides the tension member along a path between the guide and the tightening mechanism. Tensioning of the tension member pulls the guide toward the tightening mechanism. The strap system includes one or more strap guides, a first end that is coupled to the tightening mechanism, a second end that is coupled with the guide, and a strap body positioned between the first and second ends. The strap body is disposed through the one or more strap guides so that at least a portion of the strap body overlaps on itself and so that the strap body forms a loop between the first and second ends. The method also includes tensioning the tension member via the tightening mechanism to pull the guide and second end of the strap system toward the tightening mechanism to reduce a diameter of the loop and thereby effect constriction of the strap system about an article that is disposed within the loop.

### BRIEF DESCRIPTION OF THE DRAWINGS

The present invention is described in conjunction with the appended figures:

FIG. 1 illustrates an embodiment of an orthopedic brace.

FIGS. 2-3 illustrate an orthopedic brace being fit over a wearer's leg.

FIG. 4 illustrates the dynamic adjustment capability of a brace having a closure or lacing system as described herein.

FIG. 5 illustrates an embodiment of a strap that may be used with a closure system to close and/or tighten a brace.

FIGS. 6A-J illustrate embodiments of straps that are operable with a reel based closure system to close or tighten an article about a limb.

FIGS. 7A-B illustrate embodiments of straps having various strap configurations.

FIG. 7C illustrates an exemplary strap and reel based closure system configuration for a knee brace.

FIG. 8 illustrates various means for connecting a guide and lace system with a strap and/or to a brace.

FIGS. 9A-C illustrate various other strap features and/or strap configurations.

FIGS. 10A-E illustrate an embodiment of a universal strap that may be fit to a brace in a left or right configuration.

In the appended figures, similar components and/or features may have the same numerical reference label. Further, various components of the same type may be distinguished by following the reference label by a letter that distinguishes among the similar components and/or features. If only the first numerical reference label is used in the specification, the description is applicable to any one of the similar components and/or features having the same first numerical reference label irrespective of the letter suffix.

### DESCRIPTION OF THE INVENTION

Embodiments described herein provide various closure devices that may be used with medical devices, such as braces, footwear, prosthetics, orthotics, and the like. (hereinafter a brace). These devices typically include a strap (hereinafter strap or over and back strap) that is pulled over an open region of the brace and coupled with the brace to allow a user to easily don and doff the brace. The straps may also be tensioned by the user to allow the user to adjust the tightness or fit of the brace. Conventional closure straps often utilize Velcro® technology. Such straps typically attach to one end of the brace, cross the brace, loop through a D ring or other fastener, and lay back upon themselves to close the brace. Such straps often do not allow for adjustability of the brace after the strap is coupled to itself to close the brace. Thus, the user typically is required to undo the strap to adjust the fit of the brace. Moreover, the Velcro components may become dirty or soiled and lose some of the ability to tightly close the brace.

The straps described herein greatly improve closure technology by offering quick and patient friendly means of closing a brace about a body part. In many embodiments, the use of Velcro components is eliminated entirely so that the straps maintain a clean, dirt-free appearance and do not wear out over time. Further, many of the straps described herein do not stick together, which is a common problem of conventional Velcro straps. The straps also provide an improved user interface that allows for simple brace adjustment (i.e., brace tensioning and loosening). In addition, the user is often able to close the straps and tension the brace with a single hand.

For convenience, the disclosure will focus mainly on braces, although it should be realized that the embodiments described herein (i.e., the closure device and/or other devices) may be used with virtually any type of medical device and/or apparel, garment, or other structure. For example, the embodiments (i.e., closure and other devices) may be used on shoes, boots, gloves, hats, medical devices, goggles, glasses, protective gear used in sports, and the like.

In additional, for convenience in describing the embodiments, the disclosure generally describes the devices, or components thereof, being closed via a reel or dial mechanism. The reel or dial mechanism typically closes the device, or components thereof, by tensioning a lace. As described herein, a knob assembly is typically twisted to wind a lace within a spool housing and onto a spool. However, although the disclosure generally describes the closure devices, or components thereof, using a knob assembly, it should be realized that any tightening mechanism may be used and the disclosure is not limited to embodiments that only use such a knob assembly.

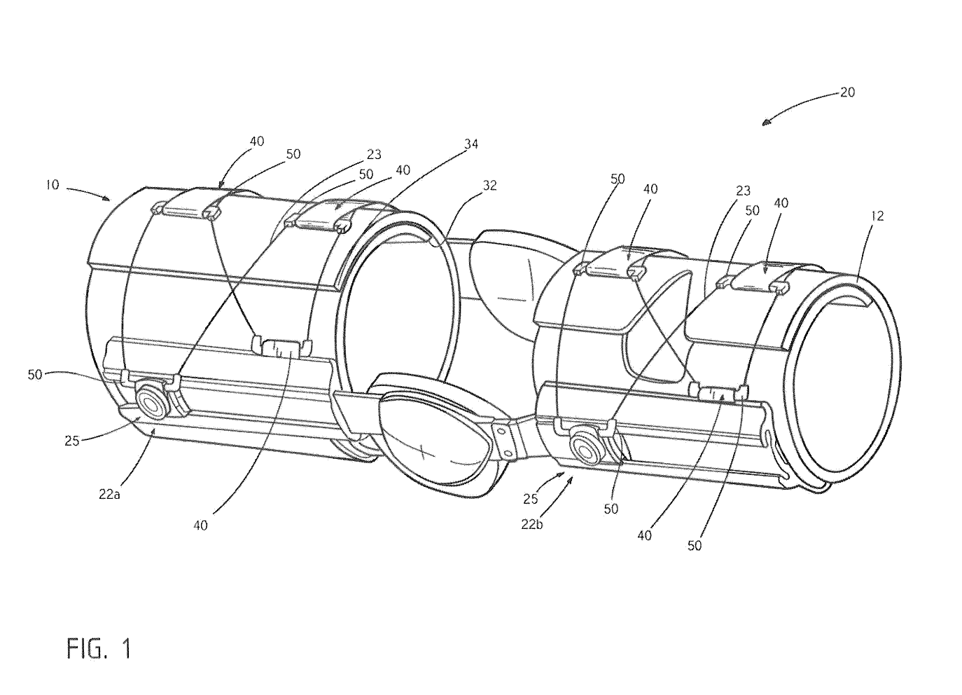

Before describing specific details of the various embodiments, a general description of a brace and closure device or system will be provided. Referring to FIG. 1, illustrated is an embodiment of an orthopedic brace 20. The orthopedic brace 20 generally comprises a knee brace that is tightened around a wearer's leg such that the knee brace substantially surrounds and protects the wearer's knee. Brace 20 may be tightened using a lacing configuration comprising two lacing systems 22a, 22b. The orthopedic brace of the illustrated embodiment is particularly concerned with relieving and/or supporting the knee joint. Although this illustrated embodiment shows the lacing systems applied to knee braces, it is to be understood that the principles discussed herein are readily applicable to any of a variety of orthopedic braces, including ankle braces, wrist braces, foot braces, elbow braces and many other types of orthopedic braces well known to those of skill in the art.

In some embodiments, the lacing configuration of closure system comprises two distinct lacing systems 22a, 22b. In some embodiments, each lacing system 22 includes a lace or cable 23 that is threaded through portions of the orthopedic brace and attached at opposite ends to a tightening mechanism 25 or reel assembly, which includes a control such as a lever, crank or knob assembly, which can be manipulated to retract the lace 23. In addition, the reel assembly 25 may comprise a mechanism of release, such as a button or lever, for disengaging the reel assembly 25, to permit the lace 23 to be withdrawn freely. In other embodiments, the reel assembly 25 may be pulled upward to allow an internal spool to spin and the lace to be pulled freely. In yet another embodiment, the reel assembly 25 may be unwound (e.g., counterclockwise) to release the spool and allow the lace to be pulled, or to unwind the lace. As shown in FIG. 1, the lace 23 may be threaded in a crossing pattern along a generally forward-facing portion of the brace 20, between two generally parallel rows of side retaining members or straps 40. In another embodiment, the lace 23 may be threaded or run laterally across the brace 20. The straps 40 may consist of a strip of material attached to the brace 20 so as to define a space in which guides 50 are positioned. The lace 23 slides through the guides 50 during tightening and untightening of the lace 23. A more thorough description of the brace 20 and lacing systems, 22a & 22b, is provided in U.S. Pat. No. 8,277,401, the entire disclosure of which is incorporated by reference herein.

The orthopedic brace 20 shown in FIG. 1 is constructed to fit a wearer's leg. The upper cuff 10 is formed to fit the wearer's thigh and curves around the thigh, generally conforming to the wearer's musculature. The lower cuff 12 is similar in construction to the upper cuff 10, and is formed to fit and curve around the wearer's calf. In some embodiments, the upper and lower cuffs 10, 12 are formed from a relatively lightweight, breathable material. In some embodiments, the cuffs 10, 12 are manufactured from a cloth, fabric, or foam-like material, or a thermoformable or non-thermoformable plastic material as would be well-known to those skilled in the art.

As shown, each of the cuffs 10, 12 are generally formed from a single piece of material that is wrapped around itself, forming two ends 32, 34 that are drawn towards each other and, in fact, may overlap. Although the ends 32, 34 are shown in an overlapping position, it should be understood that these ends might also be sized to be separated by some distance when the orthopedic brace 20 is tightened. Generally, the lace 23 may be tensioned to draw the ends 32, 34 past each other and thereby tighten the orthopedic brace 20 about the wearer's limbs. As is readily understood in the art, the two ends 32, 34 of brace 20 are designed to be open and fit about a patient's leg. The two ends 32, 34 are then positioned over the leg and brace 20 is tightened as described above.

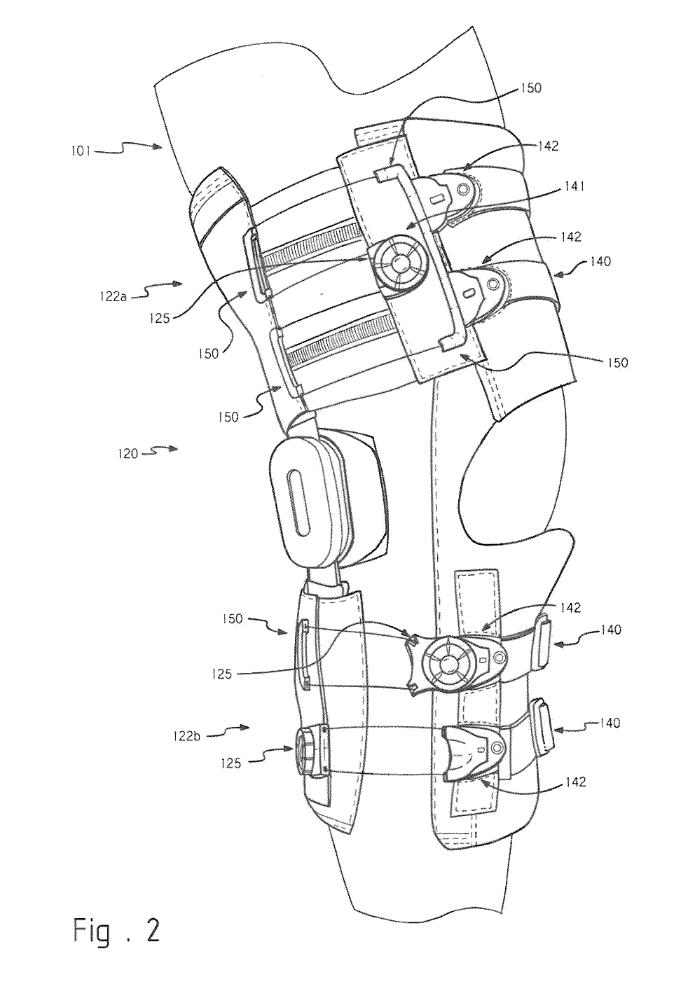

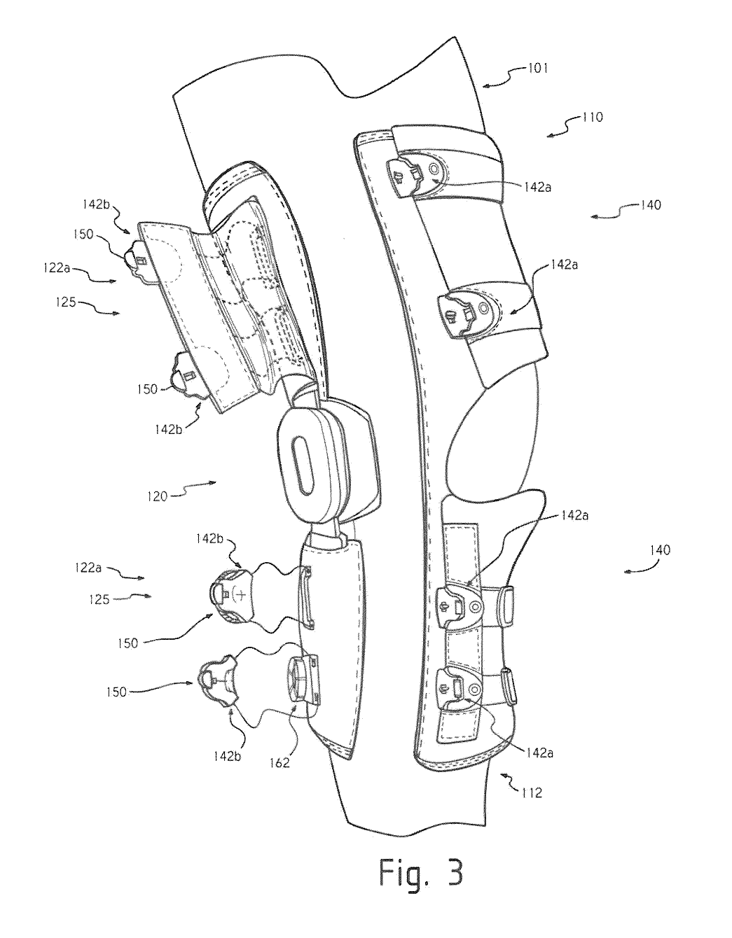

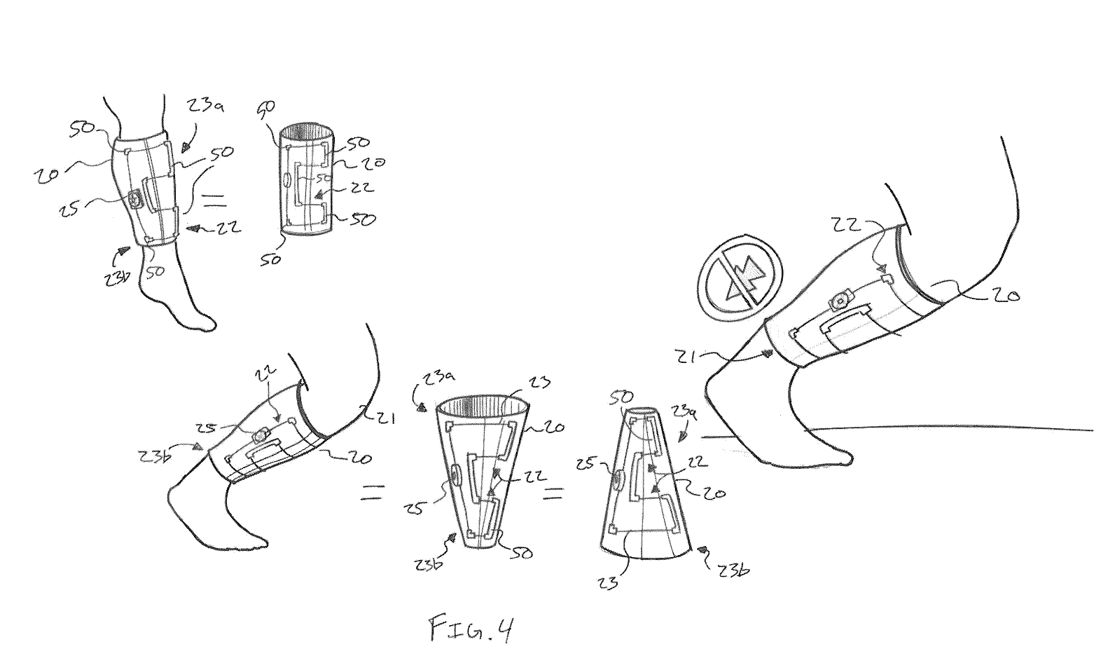

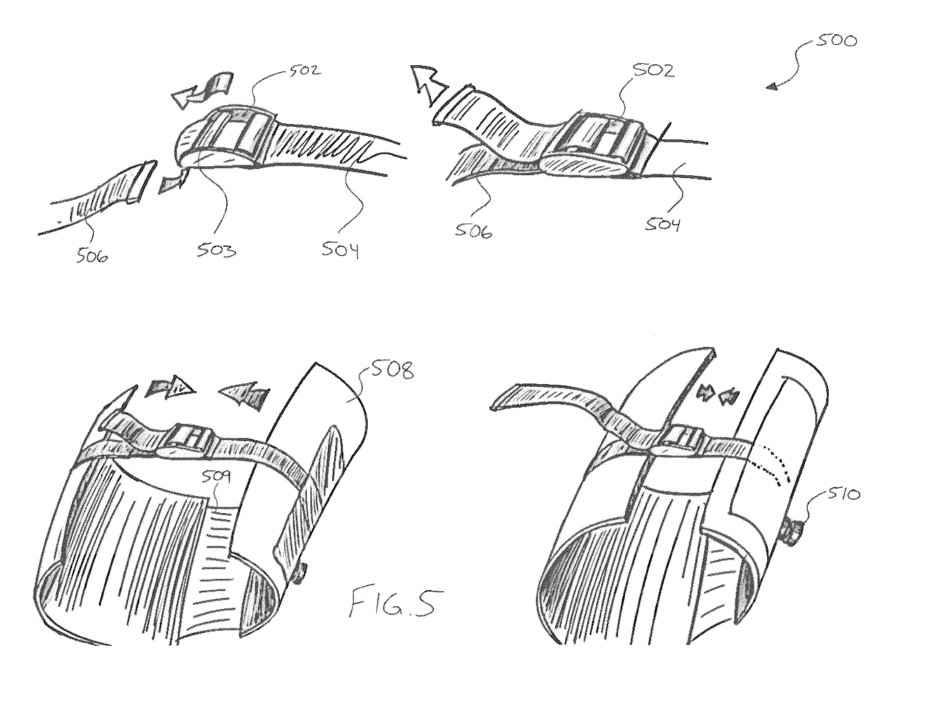

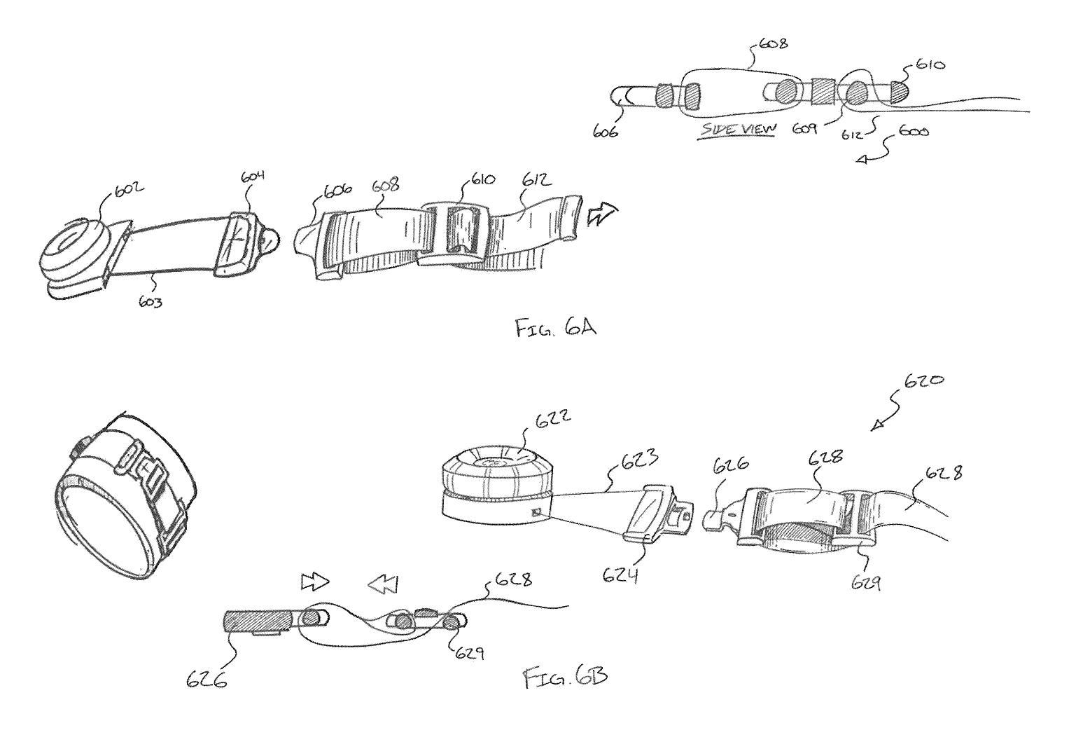

FIGS. 2 and 3 illustrate another brace 120 being fit over a wearer's leg 101. Brace 120 includes a closure system (e.g., 122a and 122b) that is described in more detail in U.S. Pat. No. 8,277,401 incorporated by reference herein. Brace 120 also includes a rough adjustment feature that permits further opening of the brace 120 to facilitate attachment of the brace 120 to a wearer's leg 101, while still providing the reel assembly 125 for final tightening. The rough adjustment feature may be variable length retaining members 140 that allow brace 120 to fit a wider variety of wearers' legs. In one embodiment, the variable length retaining member 140 includes adjustable straps. In other embodiments, a panel 141, such as those described herein, may be used. The panel 141 may be coupled with the reel assembly 125, such as a knob assembly and lace, to provide gross or macro adjustment of the brace 120. In some embodiments, retaining members 140 are configured to be releasably engaged with guides 150 opposite the reel assembly 125. The engagement may be by way of a quick release mechanism 142, for example the detachable guides described herein. In other embodiments, Fastex® buckles (not shown), Velcro® or other similar mechanisms known to those of skill in the art may be used. As shown in greater detail in FIG. 3, each quick release mechanism 142 may include a female component 142a and a male component 142b that are coupled over the wearer's leg 101 to allow brace 120 to be donned and doffed. Exemplary embodiments of male and female components, 142b and 142a, are described in the applications incorporated herein by reference. In some embodiments, the female component 142a may be attached to the guide 150 while the male component 142b is attached to the retaining member, though the arrangement of components may be switched as needed. The opposite end of the retaining member 140 may be attached to the brace such that tension in the lacing system 122 causes tension on the retaining member 140 when the quick released mechanism 142 is engaged, thereby compressing the cuffs around the wearer's limb.

Closure system 122 may include additional gross adjustment features in combination with the quick release mechanism 142 to provide a rough or gross adjustment of the closing pressure of the brace 120 prior to use of the reel assembly 125. For example, the closure system 122 may include ladder locks (e.g., Fastex Slider®) which allow the retaining members 140 to be lengthened or shortened as needed. Though shown with two retaining members 140, as with the other embodiments disclosed herein in some embodiments, the number of retaining members 140 may vary. In some embodiments, three, four, five, six or more retaining members 140 may be desirable.

FIG. 3 shows one embodiment of the brace 120 in a partially open configuration. The quick release mechanism 142 have been disconnected leaving the guides 150 attached to the brace and releasing one end of the retaining member 140. To remove the brace 120, the user may then open the cuffs 110, 112 and slide the brace from the user's leg 101. Prior to releasing the quick release mechanism 142, the user may release tension in the closure system 122 by releasing the reel assembly 125 by, for example, pulling outwards on the knob assemblies 162.

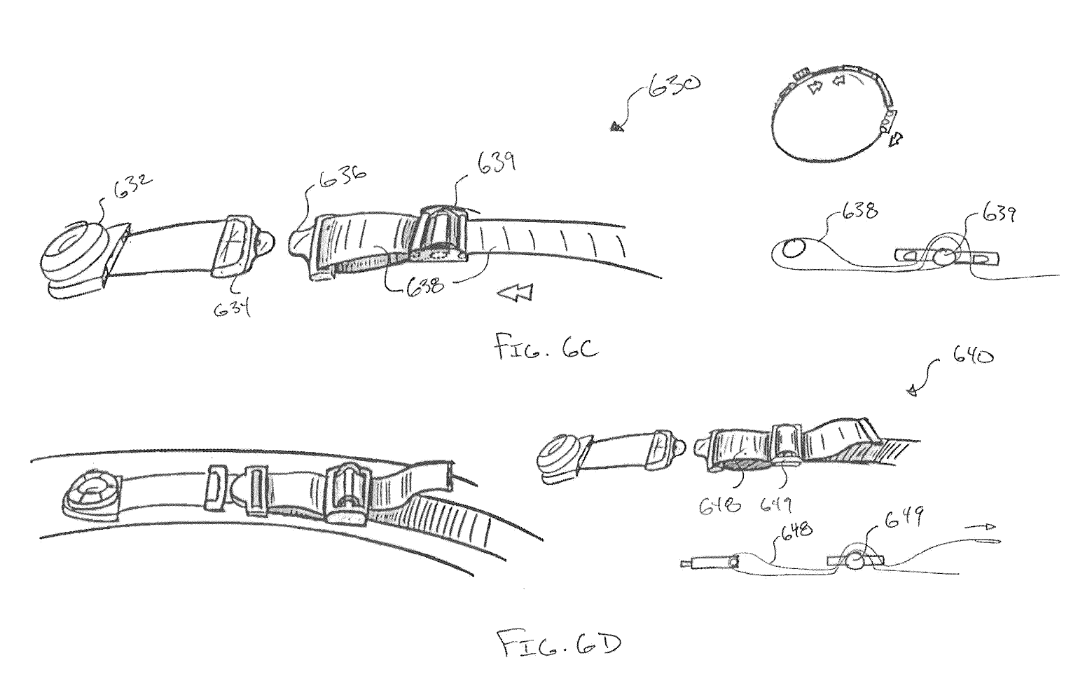

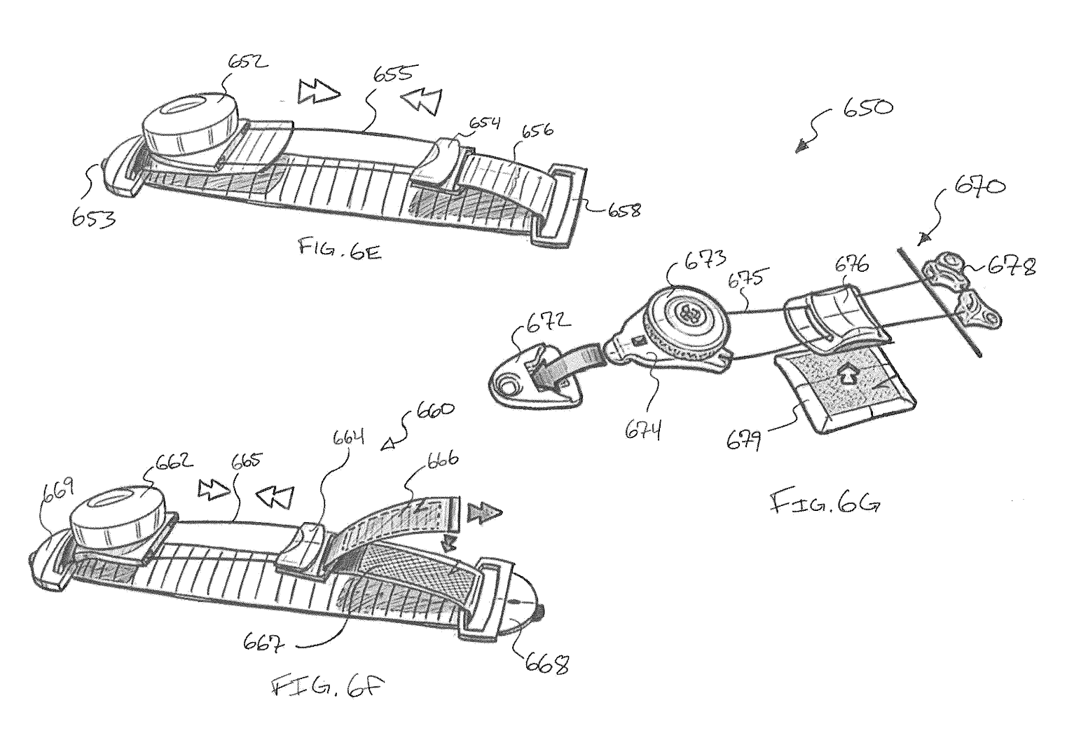

As shown in FIG. 4, one advantage of using the above described brace 20 is the increased ability of the brace 20 to fit a conical shape or an adjustable shape, such as a leg 21, arm, or any other body part of a patient. The ability of the brace 20 to fit a conical shape is provided by the lace winding system 22. As the brace 20 is fit about a conical shape (e.g., the leg 21) and the lace 23 wound via reel assembly 25, an upper portion 23a of the brace 20 contacts the conically shaped object. As the lace 23 is wound, the lace 23 adjusts until the lower portion 23b of the brace 20 also contacts the conically shaped object (e.g., the leg 21). Additional winding of the lace 23 will result in approximately equal tension throughout the lace 23, which provides a relative even pressure on the conically shaped object resulting in a good fit of the brace 20 on the conical shape.

Further, the brace 20 is able to adjust to changes in the shape of the object, such as changes in the shape of a leg 21 (or other body part) due to flexing and/or relaxing of the muscle. For example, as a leg 21 is flexed and assumes a more cylindrical shape, the lace 23 is able to slide within, or relative to, the guides 50 so that a bottom portion 23b of the brace opens or widens as a top portion 23a contracts or shrinks, or vice versa. Conventional braces typically do not adjust in this manner and as such, when a patient flexes their leg 21 (or other body part) the brace 20 is typically forced to move or migrate, such as downward against the knee or ankle. In the embodiments described herein, because the lace 23 is able to slide relative to the brace 20 and guides 50, and the brace 20 is able to adjust to changes in shape, the fit or hold of the brace about the body part is increased and migration of the brace 20 is greatly limited or eliminated.

As mentioned previously, the disclosure is directed mainly toward braces and other medical devices. However, the straps described herein may be used in a variety of different applications, including, for example: a tool bag, on a bicycle, under a bicycle's saddle to hold one or more bags or components, in a battery casing or cover, on a bag (e.g., sleeping bag), on a shoe, on goggles, on protective gear, and the like. It should be realized that many applications exists for the straps and that the straps are not limited by the embodiments described herein.

According to some embodiments, the straps described herein allow for adjustability of the brace, or other component, without completely undoing and reattaching the strap. Thus, the brace may stay in place about a patient's body part while the strap tension, and brace fit, are adjusted. Stated differently, the straps described herein provide for on the fly micro-adjustability of the brace, device, or component. Conventional braces straps (i.e., those using Velcro or similar straps) typically require the user to undo the strap, tension the strap, and then reattach the strap. Undoing the strap as required by conventional straps may cause the brace to shift on the patient's body part or possibly fall off, which may not be desired.

Some additional advantages of the straps described herein are the ability to easily close the brace and/or allow for fine adjustment of the lace tension and/or length. For example, many straps include coupling components that allow the brace to be easily coupled over the patient's body part. In contrast, Velcro or similar straps often pass through a D-ring, which forces the user to pull against the strap to tension the strap before coupling the strap on itself. In addition, Velcro often creases when set in an initial position, which may discourage further micro-adjustments. Similarly, Velcro brace systems usually have a little bit of “play” or movement after donning of the brace, which may cause unwanted loosening. The straps described herein provide greater rigidity than conventional straps.

In some embodiments, the straps provide a mechanical advantage in closing the brace when compared to conventional brace closure systems because of the knob diameter vs. the spool diameter. For example, when the reel assembly is coupled with a strap that winds around a lace guide element, the lace guide element functions as a pulley reducing the required lace tension force. Further, in some embodiments the reel assembly is coupled with two straps and therefore two lace guides, which also reduce the overall lace tension. The use of two straps allows the force to be projected into different areas of the brace. The straps may also provide increased breathability to the brace. For example, the heavy straps and webbing material used in conventional straps may be replaced by the reel assembly's lacing system. The lacing system may allow increased air access to the enclosed body part, thereby reducing the heat associated with wearing such braces.

In some embodiments, the reel assembly may include a tension indicator that visually displays the amount of tension applied by the straps. For example, the knob assembly could include a tab, counter, or dial that displays the amount of tension applied. In other embodiments, a potentiometer or other electronic device could be used to display the displacement of the strap. In other embodiments, the tension may be displayed via the laces. For example, the tension in the lace may be measured via a tensiometer. In other embodiments, the distance or displacement of the lace or straps may be measured to calculate the applied tension. For example, a measuring scale may be coupled with the lace and/or reel assembly to visually display the lace or strap displacement. Displaying the tension in this manner allows the user to repeatably “dose” the brace fit, or in other words, to repeatably fit the brace about the body part with a desired amount of pressure and tension.

In some embodiments, the reel assembly of the strap may have an automatic means of winding up lace. This may allow the strap to be easily pulled across an opening of the brace and coupled with an opposite side of the brace. The strap may be wound or pulled back across the brace's opening when decoupled from the brace. In some embodiments, the automatic means of winding up lace may be a coil spring component of the reel assembly. The coil spring component may prevent the lace from tangling when the strap is not coupled to close the brace. Having described several embodiments generally, additional aspects of the straps will become more evident with reference to the figures described below.

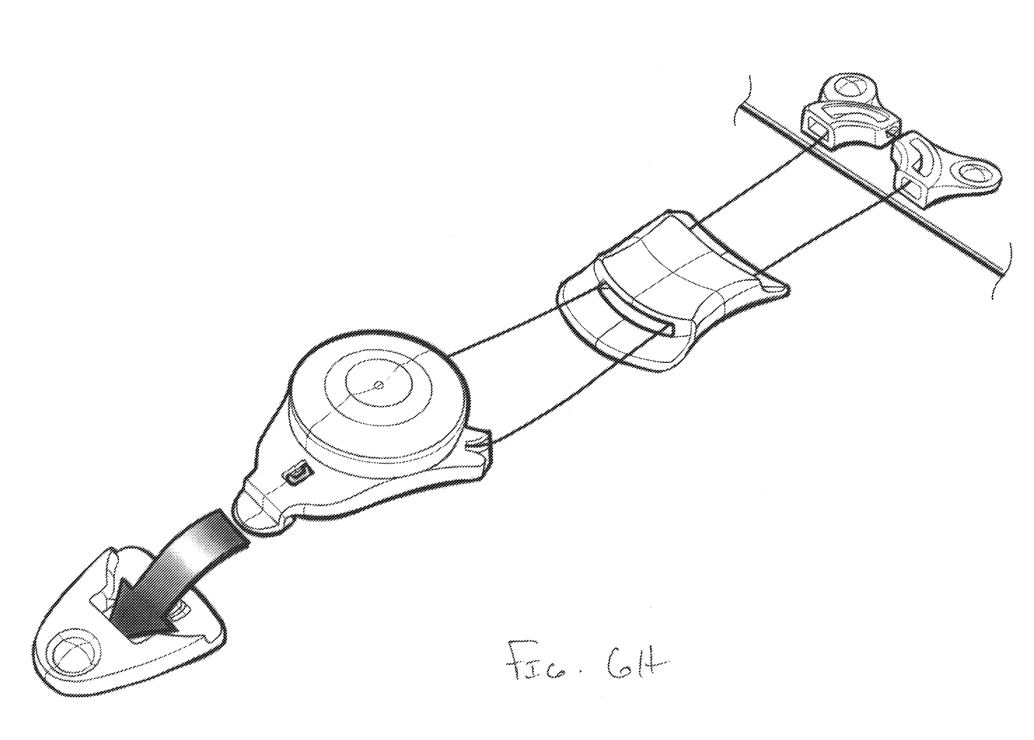

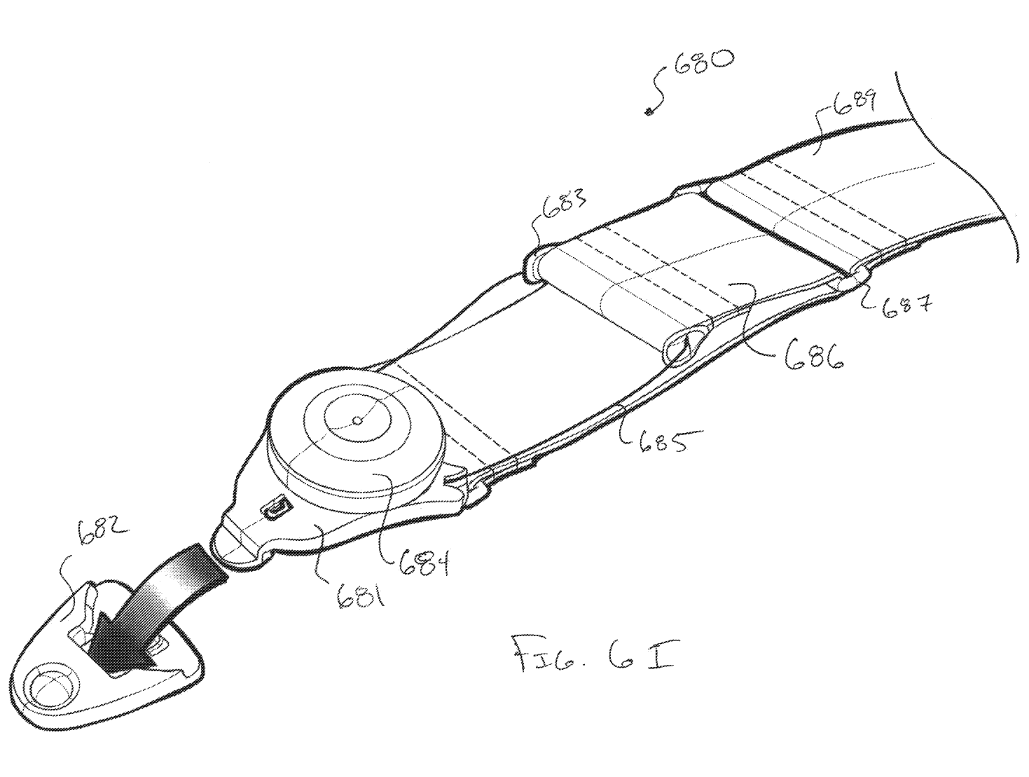

Referring now to FIG. 5, illustrated is an embodiment of a strap 500 that may be used with a reel assembly 510 to close a brace. The strap 500 includes a first strap 504 and a second strap 506 that are coupled together via a fastener 502. In some embodiments, the first strap 504 is fixedly coupled with the fastener 502, such as by stitching the first strap 504 around a rung of fastener 502 (i.e., a ladder lock fastener). The second strap 506 may wind around a separate rung 503 of fastener 502 and may be pulled against rung 503 to tension the first strap 506 and the second strap 504 to close the brace 508. The reel assembly 510 may be operated as previously described to tension lace (not shown) that extends across webbing 509, fabric, or an open portion of the brace 508. Because the brace 508 may be adjusted via reel assembly 510, the strap 500 may need to be tensioned only one time via straps 504 and 506 before all further adjustments are performed via reel assembly 510. Stated differently, the strap 500 may only be required to be tensioned once to close the brace 508 about a patient's body part and adjust to their particular size and all subsequent adjustment of the brace 508 may be performed via reel assembly 510.

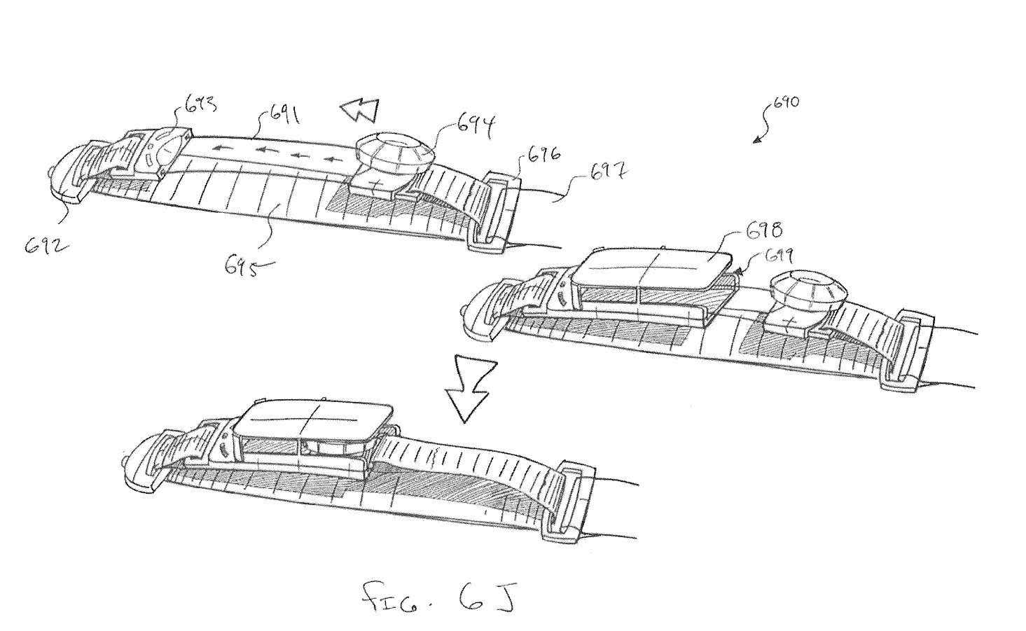

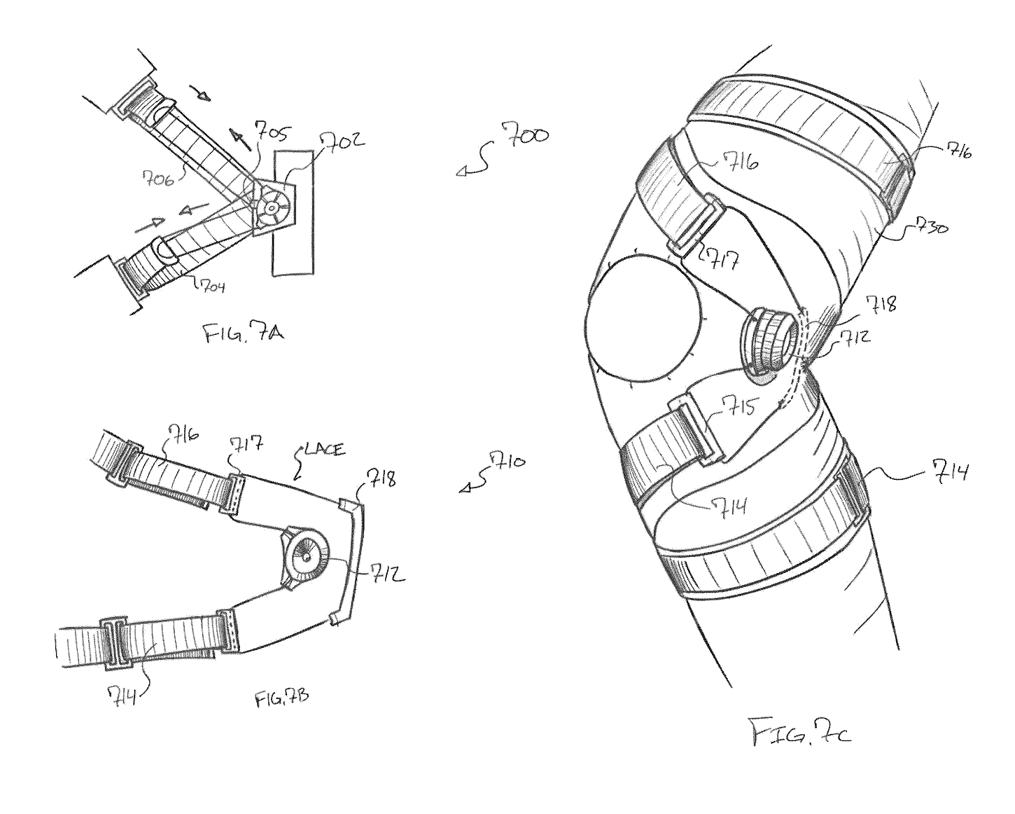

Referring now to FIG. 6A, illustrated is another embodiment of a reel system 600. Reel system 600 includes a reel assembly 602 as previously described. The reel assembly 602 is coupled with a detachable guide 604 via lace 603. Detachable guide 604 is removably couplable with component 606, which is connected to strap 608. In one embodiment detachable guide 604 comprises a female component that is couplable with a male component 606, or vice versa, as described in U.S. Provisional Application No. 61/722,022, the entire disclosure of which is incorporated herein by reference. In some embodiments, lace 603 is inserted through tubing or an aperture of guide 604. In other embodiments, lace 603 may be placed over or around an open channel of guide 604 so that the lace 603 is removable from guide 604.

Strap 608 is further coupled with a fastener 610 (e.g., ladder lock) such as the fastener previously described in FIG. 5. A second strap 612 may wind around a rung 609 of fastener 610 to allow strap 612 to be tensioned to initially close the brace or to provide further macro adjustment of the brace. Subsequent adjustment of the brace may be performed via reel assembly 602 and lace 603 as described herein.

The brace may be easily donned and doffed without tensioning strap 612 since the strap 600 may be uncoupled via guide 604 and component 606. Stated differently, strap 612 may be initially tensioned to close the brace and any subsequent donning and doffing of the brace may be accomplished by uncoupling guide 604 and component 606. Thus, the overall fit of the brace may be initially set with strap 612, after which strap 612 may remain virtually unused. In some embodiments, strap 612 may be fixedly coupled to itself or to another component so that further adjustment of strap 612 is prevented. Guide 604 and component 606 may be configured to remain coupled while lace 603 is under tension. Thus, to uncoupled guide 604 and component 606, the tension in lace 603 may be removed via reel assembly 602.

In some embodiments reel assembly 602 may include a coiled spring, such as a clock spring, or other component, that automatically winds the lace 603 within a spool housing of the reel assembly 602. The coil spring may pull guide 604 toward reel assembly 602 when guide 604 is uncoupled from component 606. The coil spring may also allow guide 604 to be pulled toward component 606 and coupled therewith. This feature allows guide 604 and component 606 be easily coupled and further allows the brace to be easily donned and doffed. The coil spring further manages the lace 603 to prevent the lace from twisting or entangling with nearby objects.

FIG. 6B illustrates another embodiment of a strap 620. Strap 620 includes a reel assembly 622 that is coupled with a detachable guide 624 via lace 623. Guide 624 is removably couplable with component 620 via male and female components described in U.S. Provisional Application No. 61/722,022, which is incorporated herein, or via any other coupling means. Component 626 is coupled with strap 628 that passes through fastener 629 as shown. In some embodiments, a terminal end of strap 628 may be fixedly coupled with fastener 629, such as by sewing the terminal end of strap 628 around a rung of fastener 629. This configuration may allow fastener 629 to be moved proximally and distally along strap 628 relative to component 626. As with strap 600, strap 620 may be uncoupled via guide 624 and component 626 to allow the brace to be easily donned and doffed. Subsequent adjustment of the brace (i.e., the adjustment after initially closing the brace the strap 628) may be performed via reel assembly 622 and lace 623. Reel assembly 622 may further include a coil spring that automatically winds lace 623 as previously described to allow guide 624 and component 626 to be easily coupled to allow the brace to be easily donned and doffed.

FIGS. 6C & 6D illustrate additional embodiments of straps. Specifically, strap 630 includes a reel assembly 632 that is connected with a detachable guide 634 via lace. Guide 634 is removably couplable with component 636 as previously described. Guide 636 is coupled with strap 638 which passes through a fastener 639 as shown. FIG. 6D illustrates a similar embodiment having a different lacing configuration of strap 648 and fastener 649. The reel assembly of FIGS. 6C & 6D may also include a coil spring as previously described to automatically wind lace and allow the brace to be easily donned and doffed.

FIG. 6E illustrates another embodiment of a strap 650. Strap 650 includes a reel assembly 652 that is coupled with a guide 654 via lace 655. Guide 654 is connected with a first end of a strap 656, such as by sewing the first end around a rung of guide 654. Strap 656 passes a around a rung of a D ring 658 and further passes around a rung of a coupling component 653, which is spaced laterally apart from D ring 658. A second end of strap 656 is coupled with reel assembly 652 via adhesive bonding, riveting, welding, mechanically fastening, sewing, and the like. As reel assembly 652 is operated to tension lace 655, a gap between the first and second ends of strap 656 is closed to draw the brace closed. In some embodiments, lace 655 may be placed over or around an open channel of guide 654 so that the lace 655 is removable from the guide 654. Reel assembly 652 may include a coil spring that automatically winds lace 655 within a spool housing and allows the lace 655 to be pulled around guide 654. In other embodiments, lace 655 may be fixedly coupled with guide 654. In some embodiments, D ring 658 may be a male or female component that is couplable with a corresponding component attached to the brace. In such embodiments, strap 650 may be fully removable from the brace by uncoupling D ring 658 and coupling component 653 from corresponding components of the brace.

Coupling component 653 is coupled with a corresponding component (not shown) that is attached to the brace. Coupling component 653 may include a male or female component of a detachable guide system as described in U.S. Provisional Application No. 61/722,022, which is incorporated herein. As stated above, in some embodiments, reel assembly 652 may include a coil spring that automatically winds lace 655 such that when component 653 is uncoupled from a corresponding component (not shown), the first and second ends of strap 656 are drawn toward one another. The coil spring may allow the coupling component 653 and reel assembly 652 to be pulled over in opening of the brace and coupled with an opposite side of the brace so that tensioning of lace 655 via reel assembly 652 closes the brace about a patient's body part.

FIG. 6F illustrates a similar embodiment of a strap 660. Similar to strap 650, strap 660 includes a reel assembly 662 that is coupled with a guide 664 via lace 665. A second end of a strap 666 is coupled with the reel assembly 662 while a first end is insertable around a rung of guide 664. The first end of strap 666 includes Velcro 667 or another attachment mechanism to allow the first end to be tensioned and coupled to itself. In this manner, strap 666 may be used to provide macro or gross adjustment of the brace while reel assembly 662 is used to provide micro or fine-tuned brace adjustments.

In some embodiments, the Velcro portion 667 of strap 666 may be positioned on a bottom surface of the strap 666 so that the strap couples on itself underneath strap 666 (i.e., opposite of that shown) and is hidden from the patient when coupled on itself. This configuration provides the advantage of discouraging the patient from uncoupling the Velcro portion 667 of strap 666 to loosen the brace. As such, the brace may be initially fit with strap 666 and afterwards adjusted via reel assembly 662.

In some embodiments, strap 666 may pass around rungs of a first coupling component 669 and a second coupling component 668 that are each removably couplable with corresponding components that are attached to the brace. In such embodiments, the strap 660 may be fully removable from the brace by uncoupling the first and second coupling components, 669 and 668, from the corresponding components of the brace. In other embodiments, either the first or second coupling components, 669 and 668, may be fixedly coupled with the brace.

The reel assemblies shown in FIGS. 6E and 6F may be “floating reels” meaning that the reel assemblies may slide relative to the brace and away from the coupling component (i.e., 653 and 669) as the lace is tensioned via the reel assembly. FIG. 6J illustrates another embodiment of a strap 690 that utilizes a floating reel. As shown, strap 690 includes a reel assembly 694 that is coupled with a guide 693 via lace 691. The guide 693 and reel assembly 694 are coupled with a strap 695 as previously described. In turn, the strap 695 is inserted around rungs of a D-ring 696 and coupling component 692 as previously described. The reel assembly 694 is designed to “float” or move toward the guide 693 as the lace 691 is tensioned. In some embodiments, the guide 693 may be coupled with a housing 698 having an opening or chamber 699 within which the reel assembly 694 may slide as the reel assembly slides toward guide 693. In some embodiments, guide 693 may be a buckle having detachable ends. In such embodiments, reel assembly 694 may include a coil spring that automatically winds lace 691 to pull guide 693 toward reel assembly 694. In other embodiments, the reel assemblies of FIGS. 6E, 6F, and/or 6J may be fixedly coupled in place so that the reel assemblies do not float.

Referring now to FIGS. 6G and 6H, illustrated is another embodiment of a strap 670 that may be used to close a brace. Strap 670 includes a reel assembly 673 that is coupled to a male component 674 that is removably couplable with a female component 672 attached to the brace. Male component 674 and reel assembly 673 are coupled with a guide 678 via lace 675. Male component 674 and guide 687 typically include a channel or aperture through which the lace 675 is inserted. Male component 674 and guide 678 are latterly spaced apart and guide 678 is coupled with an opposite side of a brace opening so that the reel assembly 673 may be operated to close a gap between the components and thereby close the gap of the brace.

In some embodiments, reel assembly 673 may include a coil spring that automatically winds lace 675 so that male component 674 is pulled toward guide 678 when male component 674 is uncoupled from female component 672. The coil spring may also allow the male component 674 to be pulled toward the female component 672 for coupling therewith. The coil spring may also help manage the lace 675 to prevent twisting of the lace or entangling of the lace with nearby objects. In some embodiments, a component 676 may be coupled with the lace 675 between the male component 674 and guide 678 to further prevent twisting or entangling of the lace. The component 676 may be removably attachable to a protective pad 679 via Velcro and the like to prevent the component 676 from rubbing against the patient's body part and/or against the brace.

FIG. 61 illustrates another embodiment of a strap 680. Strap 680 includes a reel assembly 684 that is connected to a male component 681, which is removably couplable with a female component 682 as previously described. Male component 681 is coupled with a guide 683 via lace 685. Guide 683 is coupled with a strap 686 via sewing, adhesive bonding, riveting, mechanically fastening, rf welding, and the like. The strap 686 winds around a rung of fastener 687 and further winds around and is coupled to a rung of male component 681. A second strap 689 winds around and is coupled to a rung of fastener 687. A proximal end of second strap 689 is coupled with the brace or with a component attached to the brace. Operating reel assembly 684 tensions lace 685 to pull guide 683 toward the male component 681 and thereby adjust the fit or tension of the brace about the patient's body part.

The embodiments illustrated in FIGS. 6A-J illustrate straps having parallel lacing patterns. Stated differently, the laces of the straps do not cross atop one another. The parallel lacing patterns provide an efficient transfer of force from the reel assembly and the closure force is directed substantially in one direction. This helps prevent buckling of the brace and helps prevent unnecessary tightening. The parallel lace pattern also makes concealing or hiding the laces within a strap, such as for tending the lace. The lack of lace crossings in the parallel pattern also lowers the friction of or imposed on the lace and helps the lace lay flat on a surface of a device without twisting and the like. In addition, as stated previously, the use of straps and fasteners in the above described straps provides a mechanical advantage in closing the brace when compared to conventional brace closure systems. For example, since the straps are wound around rungs of the fastener, the fasteners function as a pulley and reduce the required lace tension force in half Thus, it is easier for a patient to tension the lace and adjust the fit of the brace.

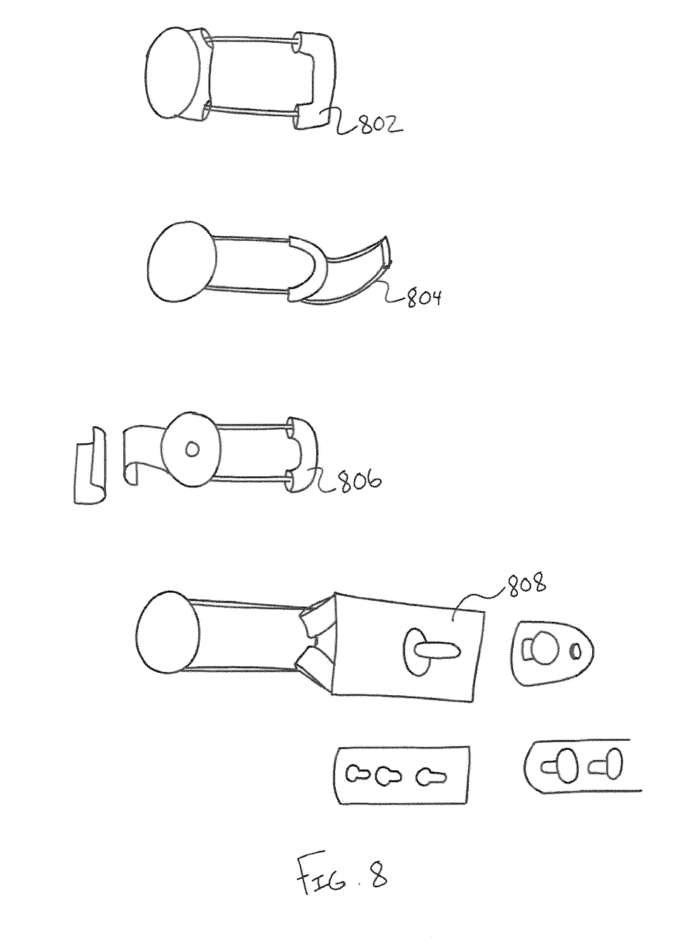

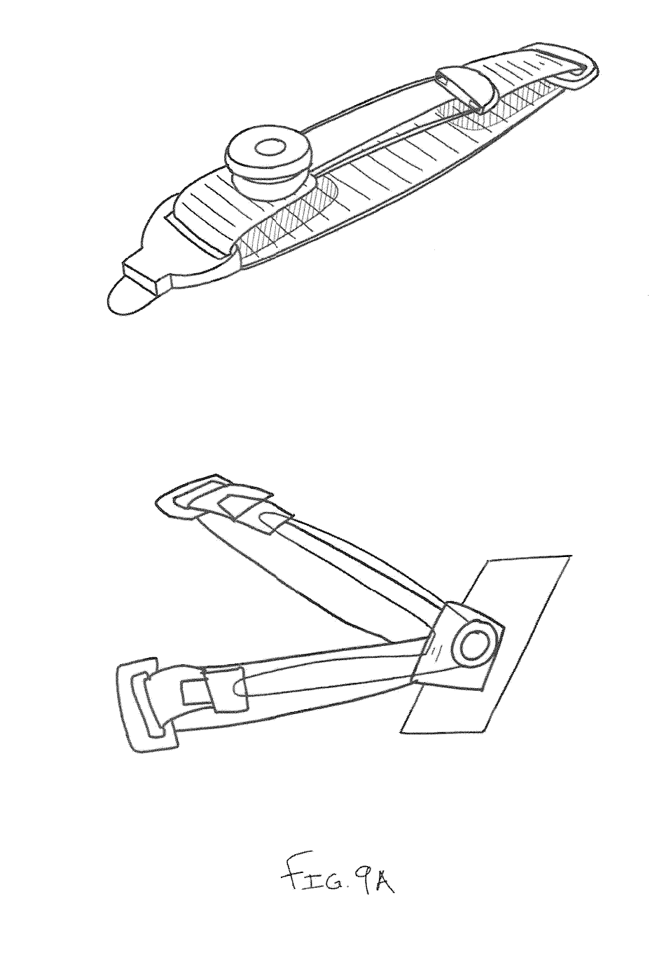

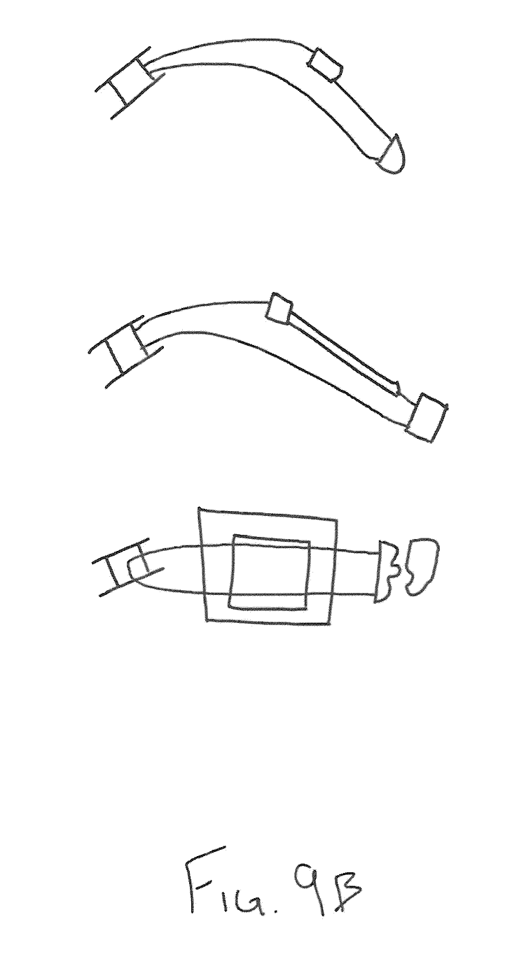

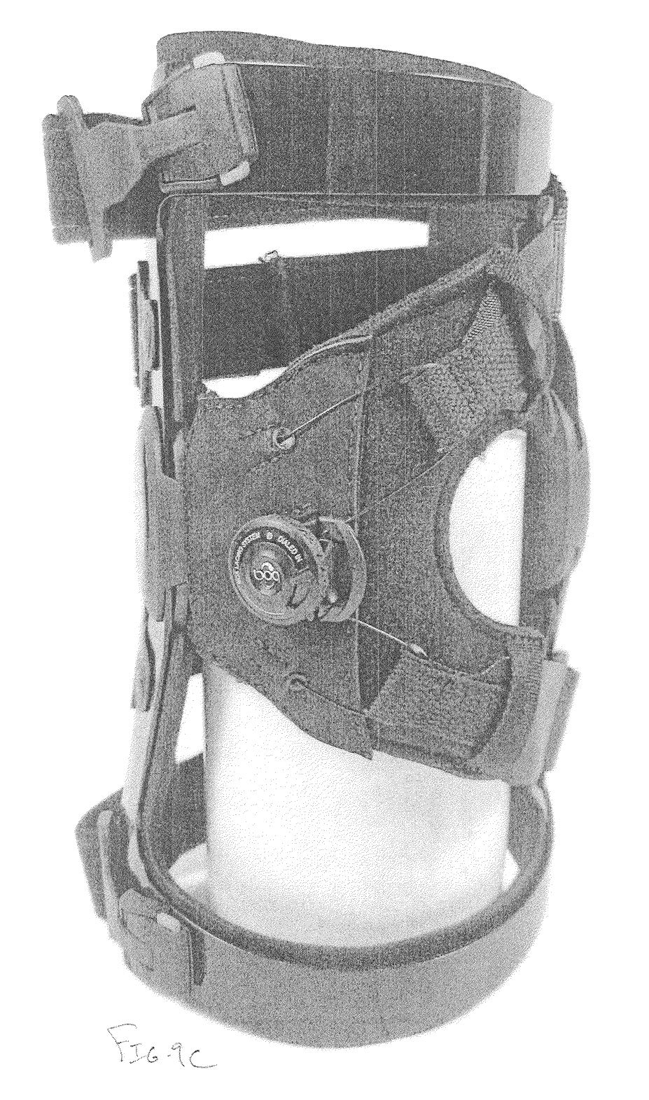

Referring now to FIGS. 7A and 7B, illustrated are embodiments of straps that include multiple strap configurations. Referring specifically to FIG. 7A, the strap 700 includes a reel assembly 702 that is coupled with a first strap 704 and a second strap 706 positioned diagonally relative to each other. The straps 704 and 706 each include guides, fasteners, and lace that are coupled with reel assembly 702 as previously described. A terminal end 705 of the lace of straps 704 and 706 is coupled near reel assembly 702. As reel assembly 702 is operated, the lace is tensioned to tension the strap and pull the guides of straps 704 and 706 toward reel assembly 702. Reel assembly 702 may be coupled with a male component (or female component) that is removably couplable with a corresponding component attached to the brace as previously described. In this manner, reel assembly 702 may be uncoupled from the brace to allow easy donning and doffing of the brace.

The use of two straps provides a mechanical advantage as previously described which reduces the required tension force in each strap in half Straps 704 and 706 are also wound around rungs of the corresponding fasteners. As such, the fasteners function as pulleys to further reduce the required tension force in each strap in half. Thus, the strap configuration shown in FIG. 7A provides a significant mechanical advantage when compared with conventional brace closure straps.

FIG. 7B illustrates another embodiment of a strap 710. Strap 710 includes a reel assembly 712 that is coupled with a first strap 714 and a second strap 716 via D-ring guides, 715 and 717 respectively. In some embodiments, the D-ring guides, 715 and 717, include channels or apertures through which lace is inserted to couple the guides to reel assembly 712. In other embodiments, the guides, 715 and 717, may include open channels over and around which the lace is placed so that the lace is removable from the guides. The first and second straps, 714 and 716, are inserted around a respective rung of the guides, 715 and 717. The first and second straps, 714 and 716, may be connected at a distal end with an additional fastener that further couples with the brace or with a strap that is attached to the brace.

The lace may have a single lace path from the reel assembly 712, through guide 715, through tubing 718, through guide 717, and back to reel assembly 712. Tubing 718 may be coupled with the brace via adhesives, sewing, mechanical fasteners, and the like. The single lace path allows the first and second straps, 714 and 716, to be equally tensioned via reel assembly 712. In some embodiments, the first and second straps, 714 and 716, may include Velcro or other attachment means to allow the straps to be lengthened or shortened as desired. In this manner gross or macro adjustment of the brace may be accomplished via the straps, 714 and 716, to initially fit the brace about the patient's body part and subsequent adjustment of the brace (i.e., micro or fine adjustment) may be performed via reel assembly 712. The Velcro may attach to itself on a rear surface that faces the brace as previously described to limit the patient's access to the Velcro attachment mechanism and encourage the patient to use reel assembly 712 to adjust the brace. In some embodiments, the straps, 714 and 716, may be fixedly coupled after the initial adjustment to prevent the user from releasing the straps. An advantage of the strap 710 configuration of FIG. 7B is the ability of the strap to dynamically adjust, for example, in instances where specific tension in each zone is desired and each strap length is individually adjusted.

FIG. 7C illustrates a specific use of a strap. Specifically, FIG. 7C illustrates a brace 730 coupled with the strap 710 of FIG. 7B. As shown, the first strap 714 is coupled with the lower portion of the brace 730 positioned adjacent the patient's calf. The first strap 714 winds around the patient's leg and couples with D-ring guide 715 near the patient's knee. The tubing 718 and reel assembly 712 are coupled adjacent and to the side of the patient's knee. The second strap 716 is coupled with the upper portion of the brace 730 positioned adjacent the patient's thigh. The second strap 716 winds around the patient's leg and couples with D-ring guide 717 near the patient's knee. Lace runs from reel assembly 712, through the guide 715 and 717, and through the tubing 718 as previously described. In some embodiments, straps 714 and 716 include the Velcro fasteners as previously described. In other embodiments other fasteners are used or the straps 714 and 716 do not include fasteners.

The strap configuration of brace 730 provides several advantages over conventional knee braces including: migration control (i.e., prevents brace 730 from sliding relative to the patient's leg), easy adjustability via reel assembly 712 and lace, the ability to telescope (i.e., stretch vertically to fit various shaped and sized patients), an increase in sizing or the ability to use a single brace for multiple patients, the ability to adjust two or more straps via a single control (i.e., reel assembly 712), and the like. Migration control is provided by the dynamic nature of lace and straps as described above that adjust to changes in shape and size as the patient flexes and moves the leg. For example, as the upper portion of the leg expands, the lace pulls through the guides, 715 and 715, and tubing 718 to allow the upper portion of the brace 730 and second strap 716 to expand. The lower portion of the brace 730 and the first strap 714 simultaneously contract about the lower portion of the leg due to the lace pulling through the guides, 715 and 717, and tubing 718. The first strap 714 also fits tightly around the gastroc to prevent downward movement of the brace 730.

Straps 714 and 716 pull around the conically shaped leg, which provide the migration control benefit and also provide angles that may benefit the fitting of that brace around the leg. An additional benefit is that some of the strap material may be reduced by using the two strap configuration, which creates more breathable space for the leg.

Although FIG. 7C illustrates the brace 730 using a single reel assembly 712 and dual strap configuration, in some embodiments the single one strap-reel assembly combinations describe in FIGS. 6A-J may be used, or a single reel assembly connected to three or more straps could be used. For example, a single strap (not shown) could be wound 360°, or some other amount, around the leg and pulled via a single reel assembly. Further, reel assembly 712 and/or tubing 718 may be removable from brace 730 to allow the patient to easily don and doff the brace. In some embodiments, the reel assembly 712 and tubing 718 may be part of or coupled with a panel that is removably couplable to the brace 730.

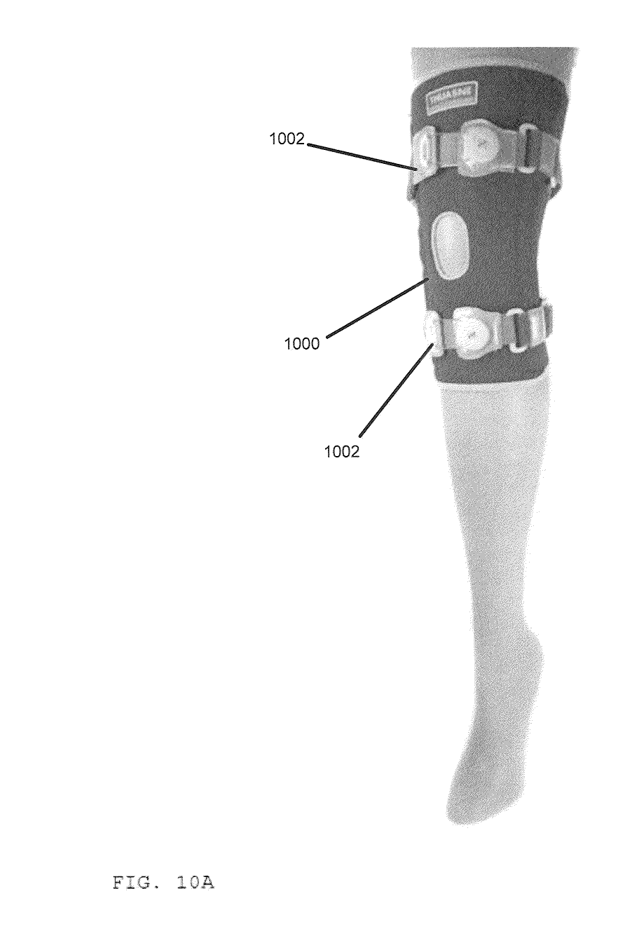

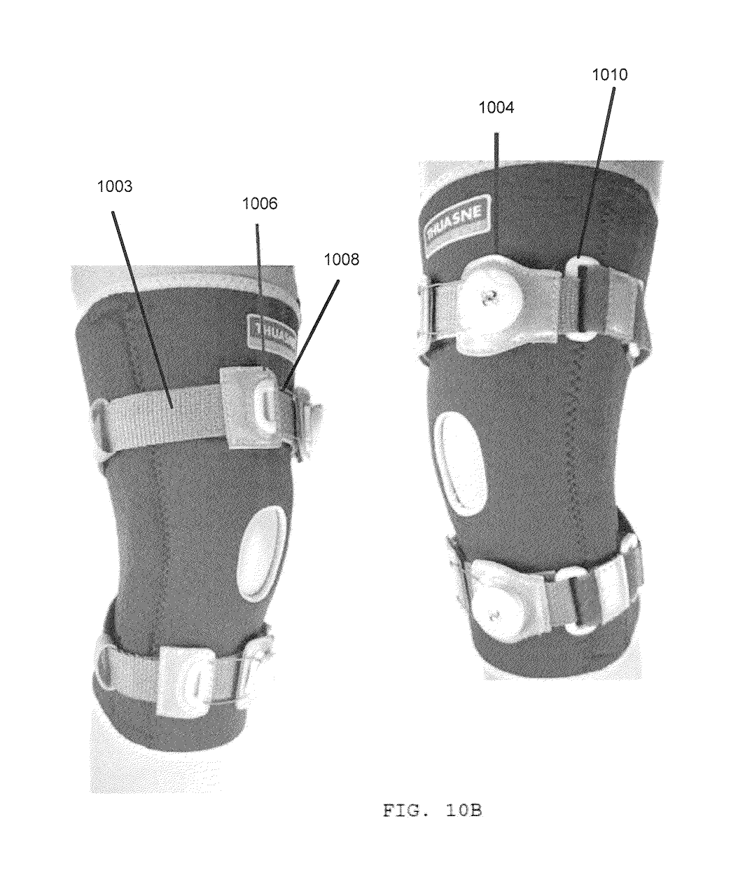

Referring to FIG. 8, illustrated are various methods in which the guide and lace may be connected to a strap and/or to the brace. These embodiments illustrate various ways in which the guide or reel may be disconnected from the brace, or a strap attached to the brace, to allow the brace to be opened for easy donning and doffing. In one embodiment, a releasable guide 802 may be coupled with the brace or with a strap that is attached to the brace. The releasable guide 802 may be uncoupled from the brace or a strap to allow the brace to be donned and doffed. In another embodiment, a pull tab 804 may be coupled with the lace. The pull tab may be pulled around and over a channel of a guide to couple the lace with the guide. Similarly, the pull tab 804 may be pulled from the guide to allow the brace to be opened. In another embodiment, the guide may be fixed 806 to the brace or strap and the reel assembly may include a hooked portion, or other mechanical means, that couple with a hook that is attached to the brace or a strap. In this manner, the reel assembly may be disconnected from the brace to allow the brace to be donned and doffed. In yet another embodiment, webbing guides may be attached to webbing 808 that includes a keyhole. A key or post that is attached to the brace or a strap may be inserted through the keyhole to removably couple the webbing and webbing guides to the brace.

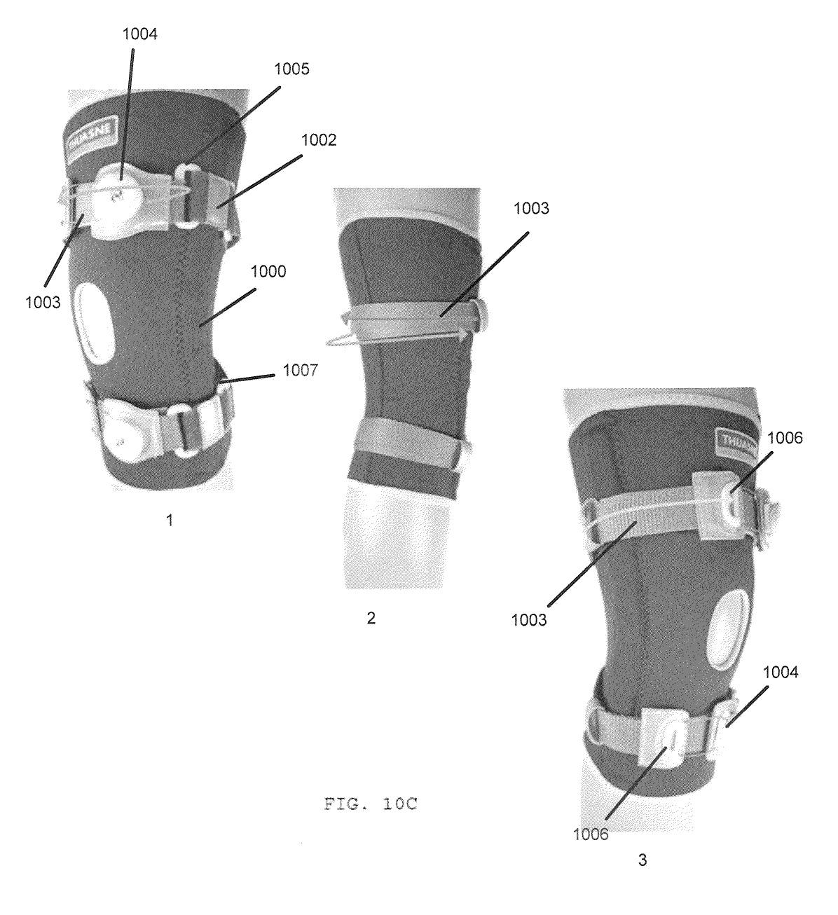

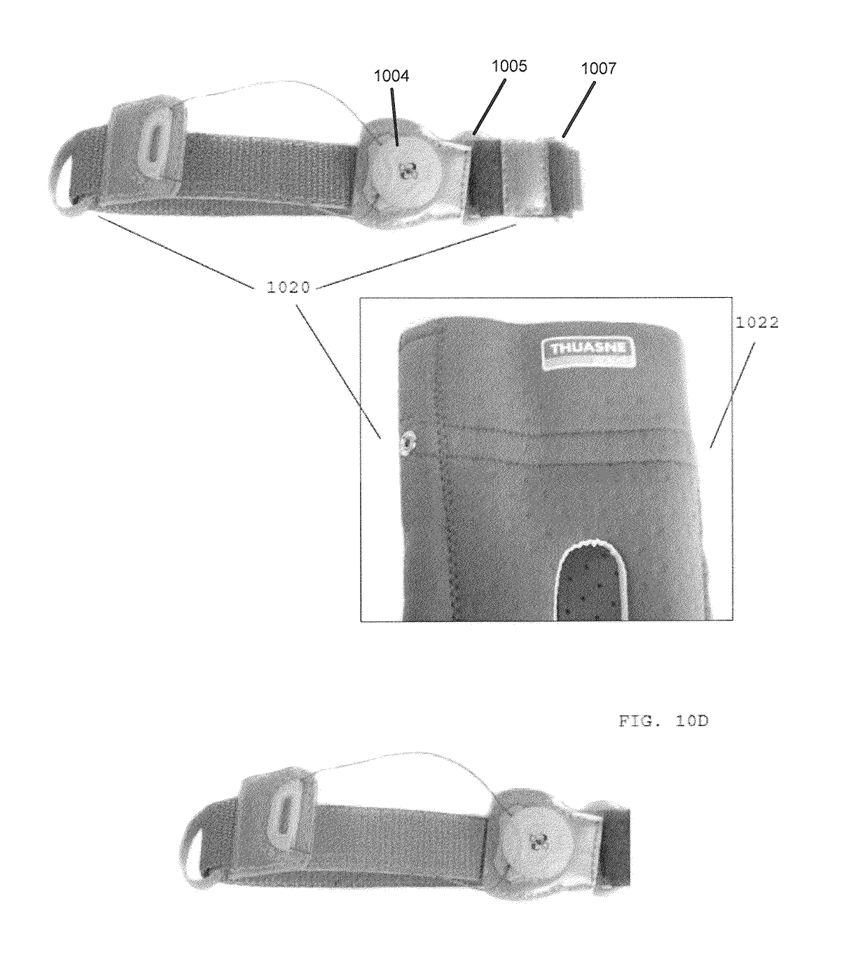

FIGS. 9A-C illustrate various other features of strap configurations.

In some embodiments, determining the location of the reel assembly on a brace may be a difficult element of brace design when applying a reel based closure device. Applying a reel assembly to a brace may introduce one or more of the following problems:

i) The reel assembly can create or increase a pressure point in certain locations such as on the tibia which can cause potential pressure or injury.

ii) Locating the reel assembly in the center of the brace may limit the closure stroke available for closing the product.

iii) Locating the reel assembly on either the medial side or lateral side may limit the brace's ability to be offered universally. This may be because people prefer to have the reel located on the lateral side. If you physically mount the reel on the brace, it forces the brace to be either a left or right brace, which increases the number of braces that a doctor must carry.

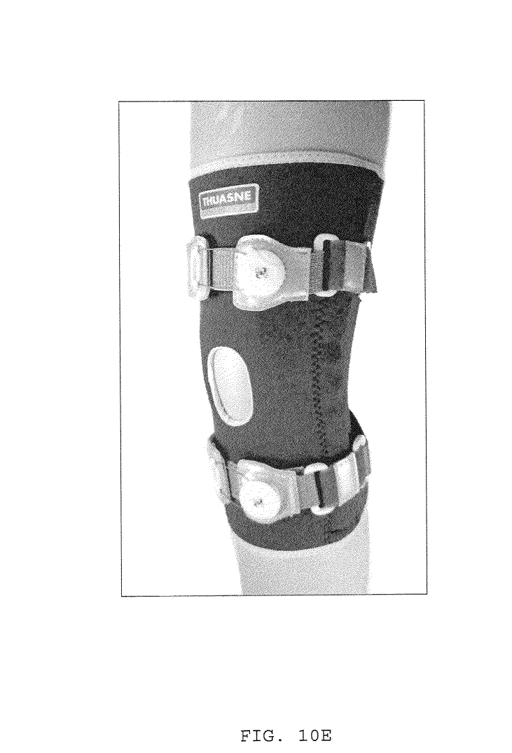

Many or all of the above problems are reduced or eliminated with the use of the brace straps illustrated in FIGS. 10A-E. The straps illustrated in FIGS. 10A-E are “universal” straps that may be easily fit to a brace in a left or right configuration. The straps of FIGS. 10A-E are also fully and easily removable from a brace. The removable aspect of the straps allow the user/physician to apply the reel assembly as a lateral mount reel no matter if the brace is worn on the left or right side. This maintains the ability of a brace to be offered as a universal brace. The reel assembly can be positioned by the user in the optimal position for their body with slight modifications in location that are possible with this strap solution. A mixture of locations for a reel assembly can be achieved if more than one reel strap exists: either all lateral, all medial, or a mixture of both locations.

An additional element of the illustrated strap design revolves around the ability for the strap to pull both posteriorly and anteriorly at the same time. If a strap is merely pulled or tensioned so as to wind around the leg circumferentially, then closure is created, but it is difficult to attach the strap to the medial and/or lateral brace stays. When a strap is tensioned in this manner, the stays can migrate out of position and compromise support, stability and fit. For example, if a strap configuration is tensioned so that the front side of the brace is pulled and a static back strap is created, complications in donning/doffing and adjustment of the brace result. In addition, the medial and/or lateral stays may be pulled off center, or out of alignment, if the back strap is not adjusted properly. Unlike conventional straps, the straps illustrated in FIG. 10A-E allow the reel assembly to pull the stays in both the posterior and anterior directions simultaneously. This feature or aspect of the straps creates the proper fit, proper support, and maintenance of the proper location of the stays.

The straps of FIGS. 10A-E overcome the conundrum of creating a universal product that utilizes reel based closure devices. The straps are left-right universal, or stated differently, can be fit to essentially any left or right brace orientation. The left-right universality of the straps reduce the number of braces that must be created, which enables lower inventory investments. The straps also remove center mount reel assembly issues including: protrusions and pressure points. The straps remove issues of a medial mounted reel assembly including: catching or snagging on the inside of the leg. The straps are physical lateral mountable without suffering the issue of part multiplication as in conventional straps.

The strap configurations of FIGS. 10A-E pull anteriorly/posteriorly to secure the stay in the proper position. This aspect or feature of the straps overcomes the problem of the stays rotating around the brace. In addition, the proper position of the stays allows the proper medial and lateral support for the joint.

The straps of FIGS. 10A-E are interchangeable and customizable. Because the straps are removable from the brace, a user has the ability to remove the strap(s) and then customize the positioning of the reel assembly. For example, the user can position the reel assemblies to be all medially positioned, all laterally positioned, or a combination of both positions.

Referring to FIGS. 10A and 10E, illustrated is an embodiment of a strap 1002 (or pair of straps) that is positioned over and around a brace 1000 that is fit about a user's leg. The strap 1002 is removably attached to the brace 1000 and is operable to squeeze or compress about the outer surface of the brace 1000 to close the brace about the user's leg. The strap 1002 may be moved longitudinally about the brace 1000 and user's leg to position the strap 1002 in a desired location for fit, closure, pressure creation or reduction, and the like.

FIG. 10B illustrates the strap 1002 fit about the brace 1000. The strap 1002 includes a strap body 1003 that may be made of a fabric or other tensionable material. Attached to the strap body 1003 is a guide 1006 that is operable to guide or route a cable or lace 1008 that is tensionable by a reel based closure device 1004. The guides 1006 have a low and inconspicuous profile about the strap 1002. The strap body 1003 is also operably coupled with one or more strap guides 1010, such as D-rings. The reel based closure device 1004 may also have a relatively low profile about the strap 1002 and may include an internal spool (not shown) about which the lace 1008 is wound. In some embodiments, about 30 cm or more of lace may be wound around the internal spool of the reel based closure device 1004. The lace 1008 is typically made of a flexible material that exhibits high fatigue durability.

FIG. 10C illustrates the strap 1002 being fit over and around the brace 1000. Specifically, the strap 1002 includes a single strap material or body 1003 that is configured to create closure all around the brace 1000 and user's leg. As shown in image 1, starting at the reel based closure device 1004, the strap 1003 loops through a ring 1005 and back over the front of the leg. As shown in images 2 and 3, after wrapping around the back of the brace and leg, the strap 1003 loops through the second ring (1007 of image 1) and all the way back toward the reel based closure device 1004, ending at the guide 1006.

As shown in FIG. 10D, in some embodiments the straps 1002 may include a button attachment 1020. The straps 1002 may be attached to the brace 1000 via an attachment mechanism 1020 located on or near the double D ring portion (i.e., 1005 and 1007) of the strap 1002 that couples with a corresponding attachment mechanism 1022 of the brace 1000. The attachment mechanism 1020 can include: lock and key buttons, snaps, magnets, velcro straps in belt loop form, and/or any other means of temporary, but secure attachment, or any combination thereof. In some embodiments, the double D ring (i.e., 1005 and 1007) could be molded as one piece with an integrated attachment mechanism molded therein. In some embodiments, the reel assembly 1004 may be affixed to an end of the strap 1002, thereby allowing the reel assembly 1004 to “float” or move relative to the strap's body and thereby allowing the reel assembly to apply tension to the strap 1002 in both directions. In other embodiments, the reel assembly 1004 may be fixed to the strap, although fixing the reel assembly to the strap may not allow the reel assembly to pull symmetrically on both stays.

In some embodiments, the double D ring piece (i.e., 1005 and 1007) may be a single molded piece with two D-rings and the button strap. In other embodiments, the D ring may be a single D ring with a snap, and a female receiver that coupled with a male coupling component, such as those described in U.S. application Ser. No. 14/071,435, entitled “Coupling Members for Closure Devices and Methods”, filed Nov. 4, 2013, the entire disclosure of which is incorporated by reference herein. The latter configuration of the strap may allow the strap to be fully openable.

In some embodiments, the strap material or body 1003 may be made of low friction materials. For example, the strap body 1003 may be low friction so that as they tighten, they do not bind on each other and cause un-even tightening. In some embodiments, the reel assembly may be attached to the end of the strap while allowing the strap to freely “float” or move through the D Ring. This configuration allows the reel assembly to pull both on the back side of the brace and the front side of the brace.

Having described several embodiments, it will be recognized by those of skill in the art that various modifications, alternative constructions, and equivalents may be used without departing from the spirit of the invention. Additionally, a number of well-known processes and elements have not been described in order to avoid unnecessarily obscuring the present invention. Accordingly, the above description should not be taken as limiting the scope of the invention.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included.

As used herein and in the appended claims, the singular forms “a”, “an”, and “the” include plural referents unless the context clearly dictates otherwise. Thus, for example, reference to “a process” includes a plurality of such processes and reference to “the device” includes reference to one or more devices and equivalents thereof known to those skilled in the art, and so forth.

Also, the words “comprise,” “comprising,” “include,” “including,” and “includes” when used in this specification and in the following claims are intended to specify the presence of stated features, integers, components, or steps, but they do not preclude the presence or addition of one or more other features, integers, components, steps, acts, or groups.

What is claimed is:

1. A strap for tightening an article about a limb comprising:
a closure system having:
a tension member;
a tightening mechanism that is configured to tension the tension member and to maintain the tension of the tension member; and
a guide that routes or guides the tension member along a path between the guide and the tightening mechanism, wherein tensioning of the tension member pulls the guide toward the tightening mechanism; and
a strap system having:
one or more strap guides;
a first end that is coupled to the tightening mechanism;
a second end that is coupled with the guide; and
a strap body between the first and second ends, the strap body being disposed through the one or more strap guides so that at least a portion of the strap body overlaps on itself and so that the strap body forms a loop between the first and second ends;
wherein tensioning of the tension member via the tightening mechanism causes the loop of the strap system to reduce in diameter, which effects constriction of the strap system about an article that is disposed within the loop.

2. A method for tightening an article about a limb comprising:
providing a strap that is configured to tighten the article about the limb, the strap including:
a closure system having:
a tension member;
a tightening mechanism that is configured to tension the tension member and to maintain the tension of the tension member; and
a guide that routes or guides the tension member along a path between the guide and the tightening mechanism, wherein tensioning of the tension member pulls the guide toward the tightening mechanism; and
a strap system having:
one or more strap guides;
a first end that is coupled to the tightening mechanism;
a second end that is coupled with the guide; and
a strap body between the first and second ends, the strap body being disposed through the one or more strap guides so that at least a portion of the strap body overlaps on itself and so that the strap body forms a loop between the first and second ends;
tensioning the tension member via the tightening mechanism to pull the guide and second end of the strap system toward the tightening mechanism to reduce a diameter of the loop and thereby effect constriction of the strap system about an article that is disposed within the loop.
